(19) United States
(12) Patent Application Publication
(10) Pub. No.: US 2004/0153035 A1
(43) Pub. Date: Aug. 5, 2004

(54) Retractable type safety syringe

(75) Inventor: Chao-Hua Shih, Taipei (TW)

Correspondence Address: DENNISON, SCHULTZ & DOUGHERTY, 612 CRYSTAL SQUARE 4, 1745 JEFFERSON DAVIS HIGHWAY, ARLINGTON, VA, 22202, US

(73) Assignee: LIFE-SHIELD PRODUCTS INC.

(21) Appl. No.: 10/747,053

(22) Filed: Dec. 30, 2003

Related U.S. Application Data
(63) Continuation-in-part of application No. 10/301,577, filed on Nov. 22, 2002.

(51) Int. Cl. A61M 5/00

(52) U.S. Cl. 604/197; 604/110

(57) ABSTRACT

A retractable type safety syringe comprises a needle set, a barrel and a plunger, wherein the needle set has a first engaging portion on the bottom thereof. The front end of the plunger has a contractible elastic piston. The piston has a second engaging portion on the top thereof to be engaged with the first engaging portion. When the piston is pushed into the injecting space of the barrel to contact the inner wall on the top end of the injecting space, and when a force is exerted to make the first engaging portion connect with the second engaging portion, the piston can restore from the deformed state to overcome the fixed connecting force of the needle set. The safety syringe has a variety of feasible shapes and structures of pistons that can suit various retractable type safety syringes.

### CROSS REFERENCE OF RELATED APPLICATION

This is a continuation-in-part application of the U.S. patent application having a Ser. No. 10/301,577 and a filing date of Nov. 22, 2002.

### BACKGROUND OF THE INVENTION

1. Field of the Invention

The present invention is related to a safety syringe of which a needle set can be drawn back into a barrel and especially to such a safety syringe that is provided with an elastic piston, which can be compressed and can restore when it is subjected to a force. The contractible elastic piston can be pushed to contact an inner wall on the top end of an injecting space in the barrel. When the needle set is drawn back after injecting, the elastic piston can help the needle set to be released from its fixed position. The action of connecting of the needle set with a plunger can be completed outside of the body of a patient; it will not increase suffering of the patient. The safety syringe is applicable to various retractable type safety syringes.

2. Description of the Prior Art

Syringes for hypodermic injection used nowadays have been widely applied in the medical field. Generally a syringe is single-use, and is discarded after use for convenience and sanitation to avoid infection of diseases through a needle set. However, the needles of the discarded syringes expose outside may stab a medical staff to make a wound or infect diseases. Wasted syringes discarded casually without well treatment may also stab people with their sharp needles; environmental personnel who treat the medical wastes may also be the victims of the needles of the medical wastes. Therefore, retractable type safety syringes have been developed to draw back the needle sets after use. The needle sets also can be hidden in barrels of syringes to reduce the volume of the syringes.

A conventional retractable type safety syringe is shown in FIG. 10, wherein a barrel (A) is formed on the front end thereof a socket (A1) for a needle set (B). The socket (A1) can be used for fixing the needle set (B) which includes a needle seat (B1) having a first engaging portion (B2) on the bottom. A plunger (C) having a pusher rod (C1) and a piston (C2) can be pushed in from the rear of the barrel (A). The piston (C2) is provided with a second engaging portion (C3) on the top thereof. The first engaging portion (B2) and the second engaging portion (C3) are left therebetween with a gap (d) in advance in order that inadvertent connecting of them with each other can be avoided. A force is required to push the pusher rod (C1) forwards in the last stage of injection of medicine, so that the first engaging portion (B2) and the second engaging portion (C3) can be connected with each other. Hence when the pusher rod (C1) is pulled backwards, the needle set (B) with a needle can thus be pulled into the barrel (A) to discard safely. There is an available product about automatic retractable type safety syringe that has needle set including a drawing-back mechanism and an activating device. After injecting, a force applied to the activating device, the needle set can be automatically drawn back into the barrel. However, in order to avoid inadvertent connecting of the pusher rod (C1) with the needle set (B) before use, the front end of the pusher rod (C1) of the syringe and the needle set (B) still are left therebetween with a gap, this makes an inaccuracy of the drawing amount of medicine. Besides, if the safety syringe does not have a stopping mechanism to avoid undue connecting of the pusher rod (C1) with the needle set (B) before use or during transportation, the syringe will become non-effective. In view of these, the above stated syringes still have the following defects:

1. Before use of the conventional syringe, the top surface of its piston and the inner top side of its barrel is left therebetween with a gap in advance to avoid inadvertent connecting of them with each other, but the gap makes an inaccuracy of the drawing amount of medicine drawn by a medical staff.

2. In the last stage of medicine injection, no matter for a manual retractable type or an automatic retractable type safety syringe, a force is required to exert on the pusher rod to make the piston completely push out the medicine and to overcome resistance against engaging and connecting or to activate an automatic needle-set drawing-back mechanism, but the force may cause pain of a patient.

In view of this, the inventor had the motive to try and develop the present invention after hard study.

### SUMMARY OF THE INVENTION

The primary object of the present invention is to provide a retractable type safety syringe provided with an elastic piston that can be compressed and can restore when it is subjected to a force. The safety syringe can help to draw or half-automatically draw a needle set back into a barrel. It is convenient for operation, effort-saving in use, and is applicable to use in an automatic retractable type safety syringe provided with a drawing-back mechanism.

The secondary object of the present invention is to provide a retractable type safety syringe that can allow the top surface of the piston to be pushed to the end to contact an inner wall on the top end of an injecting space in the barrel. A user can sufficiently exhaust air in the barrel before use, and can start to draw in medicine accurately from the zero mark on the barrel. Thereby well dosage control can be achieved.

Another object of the present invention is to provide a retractable type safety syringe of which the piston can be pushed to contact the inner wall on the top end of an injecting space in the barrel. Medicine can be completely injected before a needle seat and an engaging member above the piston are connected with each other. The plunger and the needle set can be connected with each other outside of the body of a patient by exerting a force. This can avoid increasing the pain of the patient because of drawing-back actions.

To achieve the above objects, the retractable type safety syringe of the present invention is comprised of a needle set, a barrel and a plunger. The needle set includes a needle and a needle seat for fixing the needle and having a first engaging portion on the bottom. The barrel is formed a socket for the needle set on the upper part, and an injecting space on the lower part. The needle set is connected to the socket for the needle set with a predetermined connecting force. The plunger can be pushed into the injecting space of the barrel from below, the front end of the plunger is provided with an elastic piston that can be compressed and can restore when it is subjected to a force. The piston is provided with a second engaging portion on the top thereof able to be engaged with the first engaging portion. When the piston is pushed into the injecting space of the barrel till its top surface contacts the inner wall on the top end of the injecting space, the first engaging portion has not yet been connected with the second engaging portion. When a force is exerted to press and deform the piston, the first engaging portion is connected with the second engaging portion to make releasing of the needle set from the socket for the needle set and pull the needle set into the barrel.

The present invention provides four embodiments and discloses a variety of feasible shapes and structures of pistons that can make the elastic restoring force after being compressed of the piston overcome the fixed connecting force of the needle set with the barrel.

The present invention will be apparent after reading the detailed description of the preferred embodiments thereof in reference to the accompanying drawings.

### BRIEF DESCRIPTION OF THE DRAWINGS

FIG. 1 is an exploded perspective view showing the embodiment of retractable type safety syringe of the present invention;

FIG. 2 is a schematic sectional view of the embodiment of retractable type safety syringe of the present invention;

FIG. 2A is an enlarged schematic sectional view of a part of the retractable type safety syringe taken from FIG. 2;

FIG. 2B is an enlarged schematic sectional view of a part of the retractable type safety syringe, showing that the top surface of the piston contacts an inner wall on the top end of an injecting space in the barrel. By then, the piston has not yet been deformed;

FIG. 3 is a schematic sectional view showing connecting of a first engaging portion with a second engaging portion of the retractable type safety syringe of the present invention;

FIG. 3A is an enlarged schematic sectional view of a part of the retractable type safety syringe taken from FIG. 3;

FIG. 4 is a schematic sectional view showing a needle set of the retractable type safety syringe of the embodiment of the present invention is pulled into the barrel of the syringe;

FIG. 5 is a schematic sectional view showing the piston of the embodiment of retractable type safety syringe of the present invention being formed by stacking of a plurality of elastic disk members;

FIG. 5A is an enlarged schematic sectional view of a part of the retractable type safety syringe taken from FIG. 5;

FIG. 6 is a schematic sectional view showing the piston with its middle neck portion reduced of the embodiment of the retractable type safety syringe of the present invention;

FIG. 6A is an enlarged schematic sectional view of a part of the retractable type safety syringe taken from FIG. 6;

FIG. 7 is a schematic sectional view showing the piston with a thread on its surface of the embodiment of the retractable type safety syringe of the present invention;

FIG. 7A is an enlarged schematic sectional view of a part of the retractable type safety syringe taken from FIG. 7;

FIG. 8 is a schematic sectional view showing the piston with a spring means of the embodiment of the retractable type safety syringe of the present invention;

FIG. 8A is an enlarged schematic sectional view of a part of the retractable type safety syringe taken from FIG. 8;

FIG. 9 is a schematic sectional view showing a needle and a needle seat thereof mutually connected by lure lock of the embodiment of the retractable type safety syringe of the present invention;

FIG. 9A is an enlarged schematic sectional view of a part of the retractable type safety syringe taken from FIG. 9;

FIG. 10 is a schematic sectional view of a conventional retractable type safety syringe.

### DETAILED DESCRIPTION OF THE PREFERRED EMBODIMENT

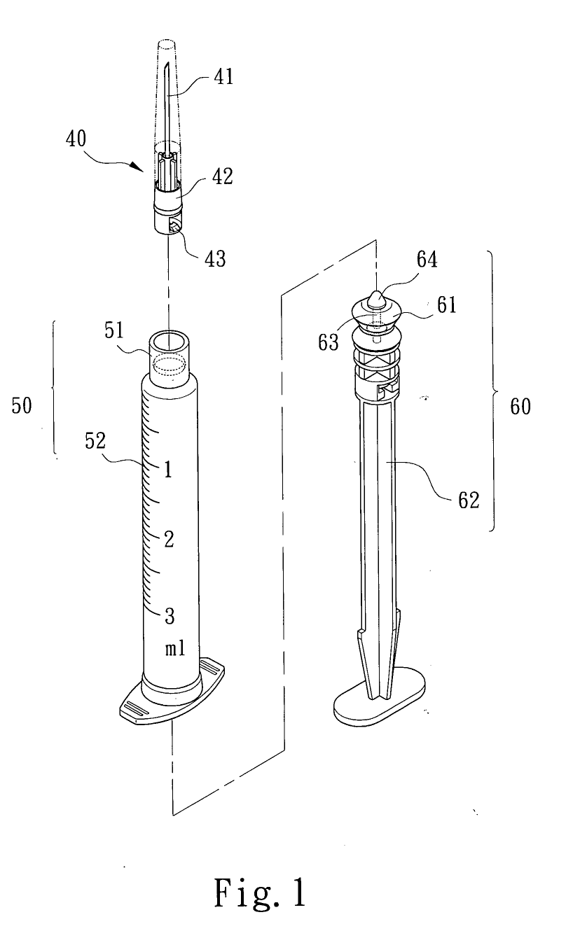

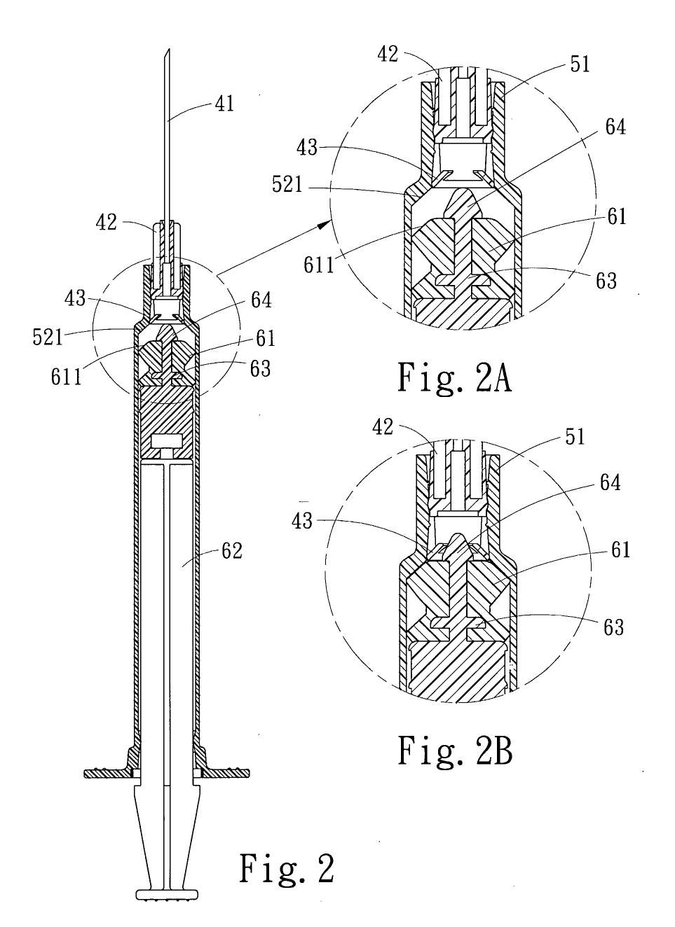

Referring to FIGS. 1, 2 and 2A, the retractable type safety syringe of the present invention comprises: a needle set 40, a barrel 50 and a plunger 60. The needle set 40 includes a needle 41 and a needle seat 42 for fixing the needle 41. The needle seat 42 has a first engaging portion 43 on the bottom. In an embodiment, the first engaging portion 43 is formed by at least a detent of which one end is fixedly provided on the inner wall of the bottom of the needle seat 42 and the other end is pended to form a contracted opening, which opened upwardly to get the form of an inverse hook. The detent 43 is hollow centrally thereof and is communicated with the needle seat 42 and the needle 41 to form a passage of medicine.

The plunger 60 can be pushed into an injecting space 52 of the barrel 50 from below, a pusher rod 62 is provided as a lower part of the plunger 60, the front end of the plunger 60 is provided centrally with a piston post 63, a contractible elastic piston 61 is slipped over the piston post 63. The piston post 63 further is provided on the top thereof with a second engaging portion 64 which protrudes out of the top surface of the piston 61. An embodiment of the second engaging portion 64 is a hook in the shape of a cone. The bottom of the second engaging portion 64 contacts the top surface of the piston 61.

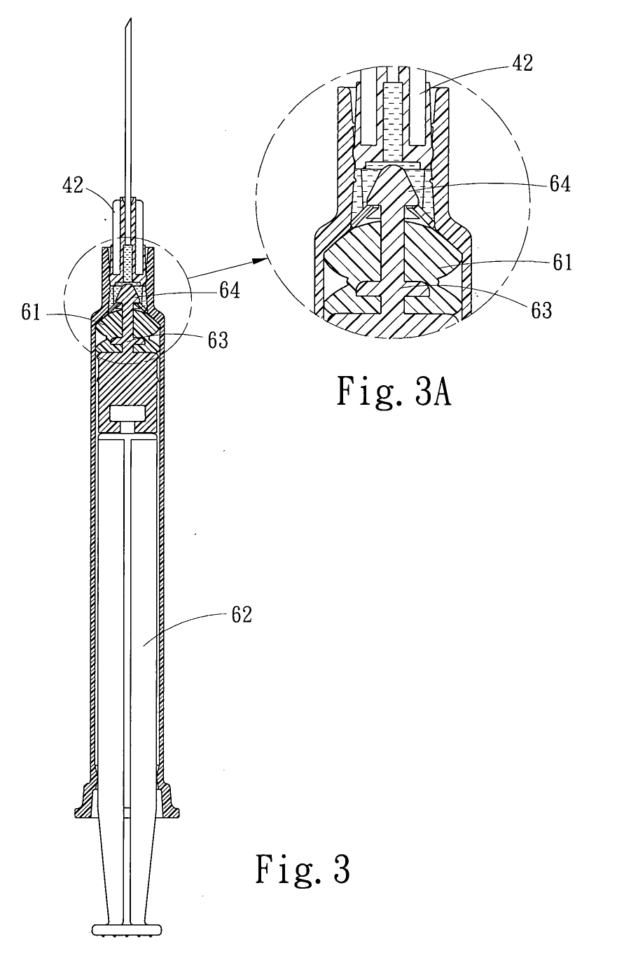

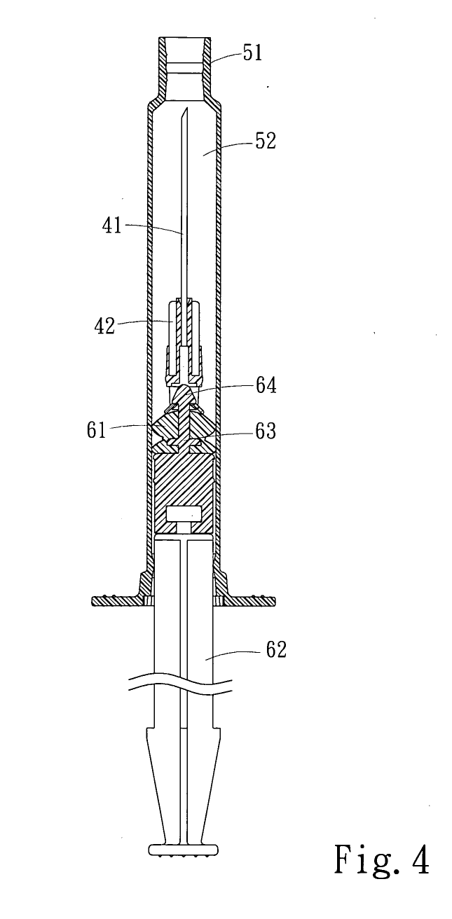

As shown in FIGS. 1, 2 and 2A, the embodiment of the piston 61 is formed from two mutually opposite elastic conic members, the peripheral round edges of the elastic conic members closely fitted with the inner wall of the barrel 50. When a doctor or a nurse is to draw in medicine, the top surface 611 of the piston 61 can be pushed to contact the inner wall on the top end 521 of the injecting space in the barrel 50. The first engaging portion 43 has not yet been connected with the second engaging portion 64 (referring to FIG. 2B), so that the user can start to draw in medicine from the zero mark on the barrel 50. During injecting the medicine, the pusher rod 62 is pushed toward the inner wall on the top end 521 of the injecting space till the top surface 611 of the piston 61 contacts the inner wall 521. The medicine is pushed out of the barrel 50; the first engaging portion has not yet been connected with the second engaging portion now. Therefore, after completion of injection, the needle set 40 is moved out of the body of a patient, and a force is exerted to push forwards the pusher rod 62. Thereby the elastically compressed piston 61 also is pressed to deform, meantime the first engaging portion 43 is connected with the second engaging portion 64 such as are shown in FIGS. 3 and 3A. When the force is removed, restoration force of the piston 61 will help the needle set 40 to be pulled back into the barrel 50. At this time, the user only slightly pulls out the pusher rod 62 rearwardly, the needle set 40 is half-automatically drawn back into the injecting space 52 of the barrel 50, such as is shown in FIG. 4. Thus the waste needle set 40 will not be exposed outside, and the purpose of the safety syringe can be achieved.

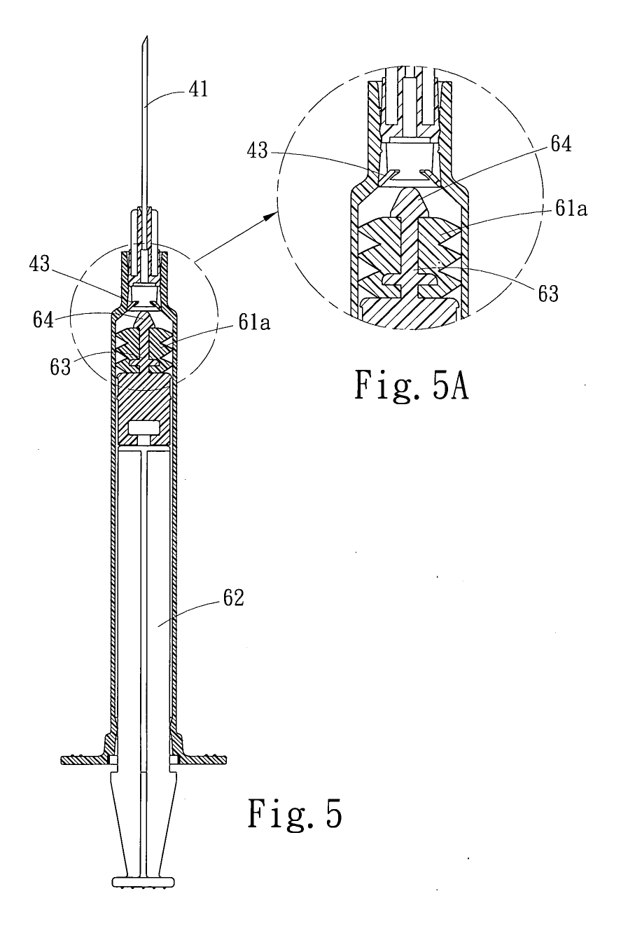

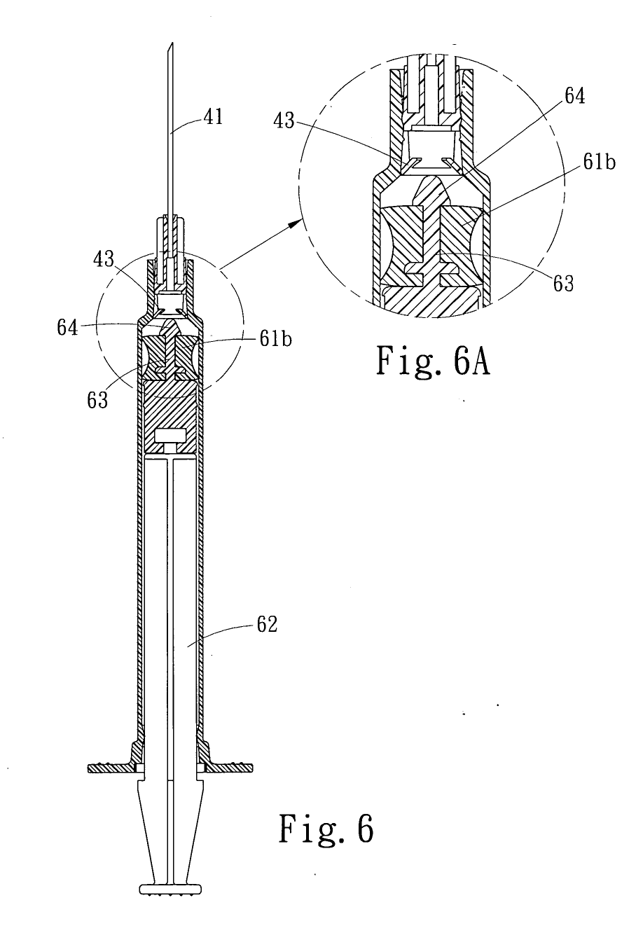

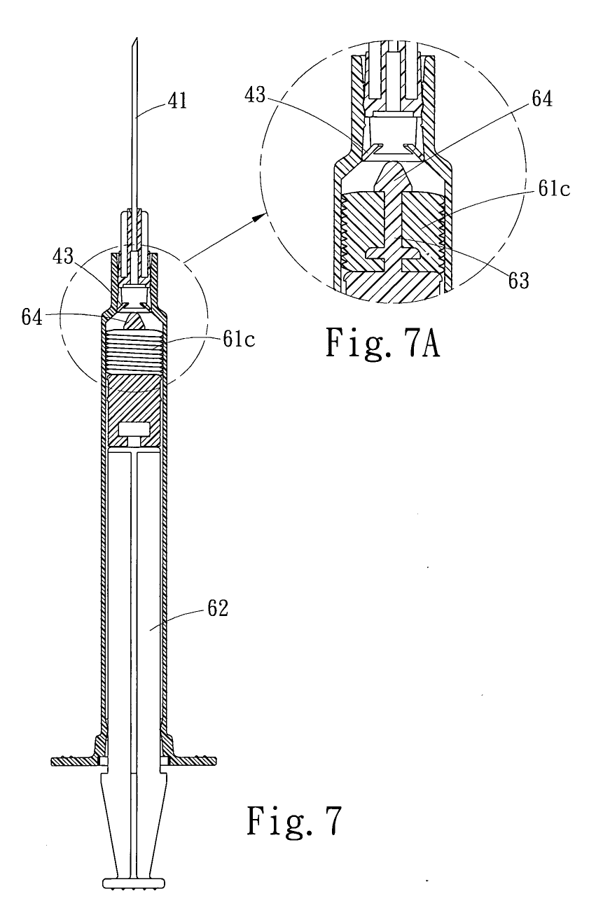

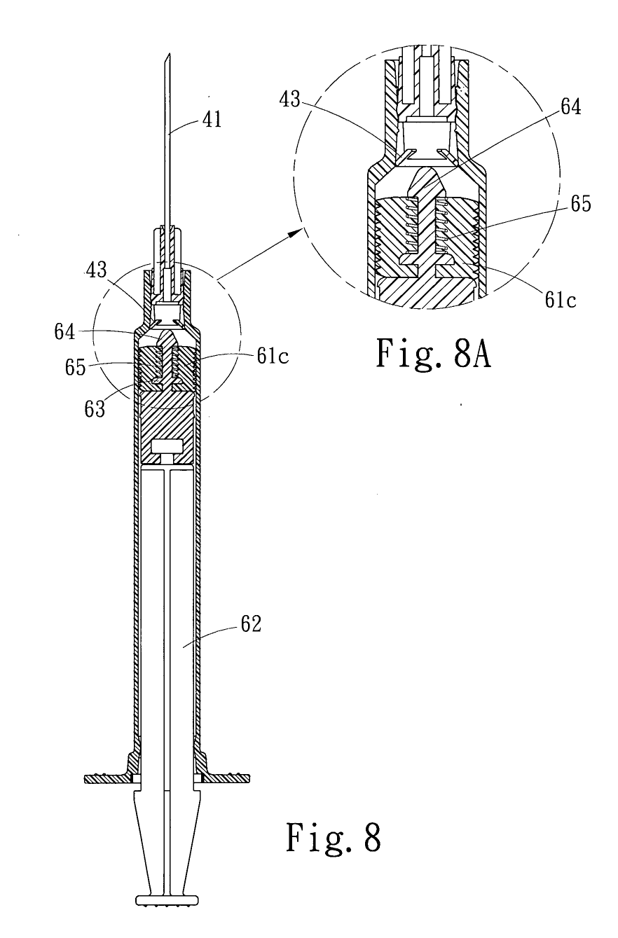

The construction of the piston can also be designed as a piston 61a at least having two elastic round-disk members stacked one over the other, such as are shown in FIGS. 5 and 5A. The piston 61b can also be designed as an elastic column-like piston 61b with its middle neck portion reduced, such as are shown in FIGS. 6 and 6A. The piston 61b can be designed as a column-like piston 61c with a thread on its surface, such as are shown in FIGS. 7 and 7A. In order to increase the elastic restoring force of the piston to widen the application scope of the syringe, a spring means 65 can be provided centrally and axially in the piston 61, 61a, 61b and 61c, such as are shown in FIGS. 8 and 8A. Surely, other similar column-like forms or different forms of the piston can be adapted. If the piston can restore after being compressed and has elastic restoring force to overcome the fixing force between the needle set and the barrel, it shall be deemed as falling in the scope of the present invention.

In practice, the compressible and restorable elastic piston of the present invention can also be applicable to those automatic retractable type safety syringes (not shown) provided each with a needle-set drawing back mechanism and an activating device, wherein the activating device can be provided on either the needle set or a front end of the plunger, and the drawing-back mechanism can be provided on either the needle set or a front end of the plunger which is not provided with activating device. When the activating device activates the drawing-back mechanism, the needle set can be automatically pulled back into the barrel by the drawing-back mechanism. The principle of function is as below: when the piston is pushed into the injecting space in the barrel till its top surface contacts the inner wall on the top end of the injecting space, the activating device has not yet activated the drawing-back mechanism now. A force is required to press and deform the piston, and then the activating device can be activated to make operation of the drawing-back mechanism. Thereby the needle set can be pulled back into the barrel by the prepared restoring force.

And more, the feasible embodiments in structural designing of the first engaging portion and the second engaging portion can be those with shapes and sizes that are mutually matched, mutually connectable or that can be mutually changed to have the same connecting effect. Such modifications or changes can be done by those skilled in this art and shall fall within the scope of the present invention directing to the first and the second engaging portions of the retractable type safety syringe of the present invention.

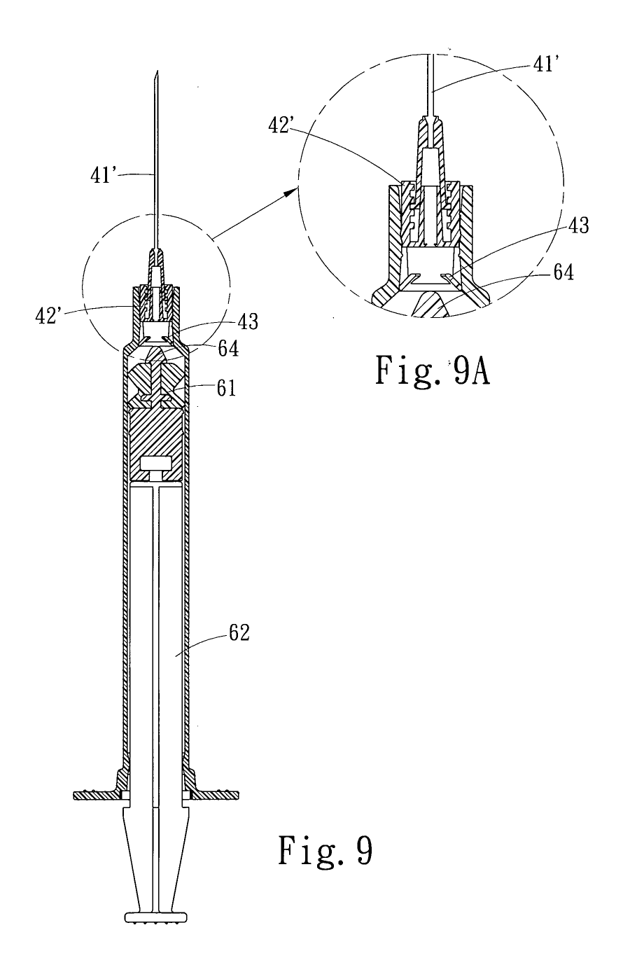

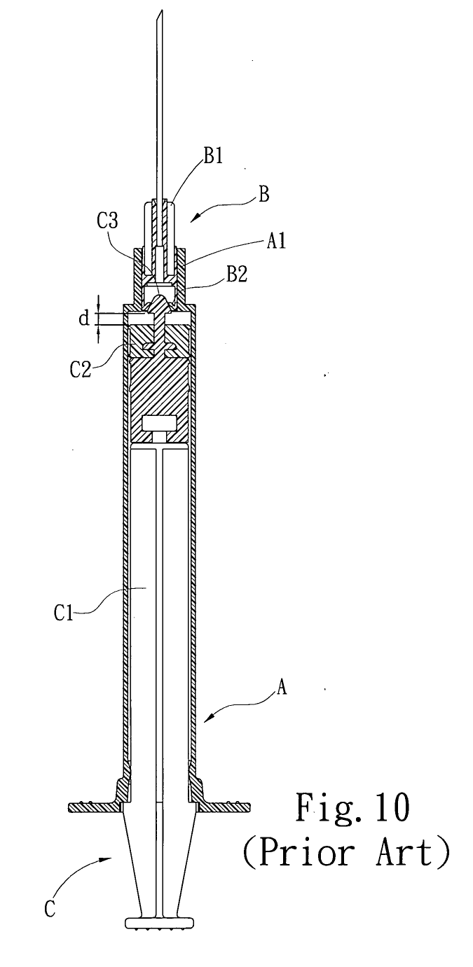

Referring further to FIG. 9, in practice, a needle 41′ and a needle seat 42′ can also be designed to have a connecting mode that allows movable assembling and detaching of them, so that the needle 41′ can be changed between the time of drawing in medicine and the time for injection. The needle 41′ and the needle seat 42′ can be mutually rotating connected (Lure Lock), or the needle 41′ and the needle seat 42′ can be mutually engaged directly (Lure Slip). FIG. 9 only shows an embodiment of lure lock of the needle 41′ and the needle seat 42′. Other needles and needle seats of different shapes and modes of connection can also be used in the half-automatic retractable type safety syringes of the present invention.

The above-disclosed structures of the present invention can be used in various retractable type safety syringes in the medical field, and has the following advantages:

1. The retractable type safety syringe of the present invention can make the elastic restoring force after being compressed of the piston overcome the connecting force between the needle set and the barrel. So that the users of the syringe can half-automatically draw the needle set back into the barrel.

2. The retractable type safety syringe of the present invention is applicable to connecting the barrel and the needle set with each other after all the medicine for injection is completely injected and the needle is extracted out of the body of a patient. This can avoid causing the feeling of pain and discomfort of the patient because of an additional exerting force.

3. Connecting of the needle set with a plunger needs a relevant force. Such a force is designed to avoid undue connecting of the needle set with the plunger before use or during transportation to render the syringe to become non-effective.

4. Before injection of the retractable type safety syringe of the present invention, the piston can be pushed to contact an inner wall on the top end of the injecting space in the barrel. So that air in the barrel can be completely exhausted, or medicine can be drawn in accurately from the zero mark on the barrel. Thereby well dosage control can be achieved.

As stated in the above disclosed, the present invention can surely achieve its expected objects to provide a retractable type safety syringe. The construction of the syringe is simple and is convenient for use; it has its industrial practical value. Having thus described my invention, what I claim as new and desire to be secured by Letters Patent of the United States are:

1. A retractable type safety syringe comprising:
a needle set, being provided on its bottom with a first engaging portion;
a barrel, being formed a socket for said needle set on an upper part, and a lower part thereof being an injecting space, said needle set being connected to said socket for said needle set with a predetermined connecting force;
a plunger, being pushed into said injecting space of said barrel from below, a front end of said plunger being provided with a piston having on the top thereof a second engaging portion adapted to being engaged with said first engaging portion, said piston being a contractible elastic means and being adapted for being compressed and adapted for restoring when it is subjected to a force;
said piston is adapted for being pushed upwardly till a top surface thereof contacts an inner wall on a top end of said injecting space in said barrel, said first engaging portion has not yet been connected with said second engaging portion now, when a force is exerted to press and deform said piston, said first engaging portion is connected with said second engaging portion, said needle set thus is released from said socket for said needle set and drawn back into said space.

2. The retractable type safety syringe as claimed in claim 1, wherein said piston is formed from two mutually opposite elastic conic members.

3. The retractable type safety syringe as claimed in claim 1, wherein said piston is formed at least from two elastic round-disk members stacked one over the other.

4. The retractable type safety syringe as claimed in claim 1, wherein said piston is an elastic column-like piston with its middle neck portion reduced.

5. The retractable type safety syringe as claimed in claim 1, wherein said piston is an elastic column-like piston with a thread on its surface.

6. The retractable type safety syringe as claimed in claim 1, wherein said elastic restoring force after being compressed of said piston is larger than a predetermined connecting force of said needle set with said socket for said needle set, thereby said syringe is formed a half-automatically retractable type safety syringe.

7. The retractable type safety syringe as claimed in claim 1, wherein a spring means is provided centrally and axially in said piston to increase said elastic restoring force and restoring action after being compressed of said piston.

8. The retractable type safety syringe as claimed in claim 1, wherein said first engaging portion is at least a detent provided on the bottom of said needle set, said second engaging portion of said plunger is a hook protruding out of said top surface of said piston, the bottom of said hook contacts said top surface of said piston.

9. The retractable type safety syringe as claimed in claim 1, wherein said needle set includes a needle and a needle seat, said needle is fixedly connected to said needle seat.

10. The retractable type safety syringe as claimed in claim 8, wherein said needle set includes a needle and a needle seat, said needle is detachably connected to said needle seat.

11. The retractable type safety syringe as claimed in claim 1, wherein said hook is provided on a front end of a piston post which is provided on a front end of said plunger for supporting said piston.

12. A retractable type safety syringe comprising:
a needle set;
a barrel, being formed a socket for said needle set on an upper part, and a lower part thereof being an injecting space, said needle set being connected to said socket for said needle set with a predetermined connecting force;
a plunger, being pushed into said injecting space of said barrel from below, a front end of said plunger being provided with a piston which is a contractible elastic means and is adapted for being compressed and adapted for restoring when it is subjected to a force;
an activating device provided on either said needle set or a front end of said plunger, and
a drawing-back mechanism provided on either said needle set or a front end of said plunger which is not provided with said activating device, when said activating device activates said drawing-back mechanism, said needle set is automatically pulled back into said barrel by said drawing-back mechanism;
said piston is adapted for being pushed upwardly to contact an inner wall on a top end of said injecting space in said barrel, said activating device has not yet been connected with said drawing-back mechanism now, when a force is exerted to press and deform said piston, said activating device is activated to operate said drawing-back mechanism, said needle set thus is drawn back into said barrel.
